(19) United States
(12) Patent Application Publication
(10) Pub. No.: US 2004/0029777 A1
(43) Pub. Date: Feb. 12, 2004

(54) Pharmaceutical preparation for taste masking

(75) Inventors: Shinji Ando, Tokyo (JP); Valentino J. Stella, Lawrence, KS (US)

Correspondence Address: SUGHRUE MION, PLLC, 2100 Pennsylvania Avenue, NW, Washington, DC, 20037-3213, US

(73) Assignee: TAISHO PHARMACEUTICAL CO., LTD.

(21) Appl. No.: 10/409,171

(22) Filed: Apr. 9, 2003

Related U.S. Application Data
(60) Provisional application No. 60/370,734, filed on Apr. 9, 2002.

(51) Int. Cl. A61K 38/00; A61K 31/192

(52) U.S. Cl. 514/2; 514/570

(57) ABSTRACT

The present invention relates to taste-masked pharmaceutical oral dosage formulations comprising complexes of a drug and polylysine or polyarginine for taste-masking, to convenient taste-masked oral dosage forms comprising the complexes and to methods of masking the taste of a drug, and preventing sublimation of the drug while providing good bioavailability.

### CROSS REFERENCE OF RELATED APPLICATION

This application claims the benefit of U.S. Provisional Application No. 60/370,374, filed Apr. 9, 2002.

### BACKGROUND OF THE INVENTION

The present invention relates to taste-masked pharmaceutical oral dosage formulations. Specifically, the invention relates to complexes of an acidic drug and polylysine or polyarginine for taste-masking drugs, to convenient taste-masked oral dosage forms comprising the complexes and to methods for masking the taste of a drug.

Oral dosage forms are by far the most commonly used drug delivery forms. Patients prefer dosage forms that are easy to swallow and have no taste or have a pleasant taste. Unfortunately, many active ingredients have a very bitter taste. This is a distinct problem if it is desirable to formulate these drugs in an oral dosage form. Various methods have been developed for masking the taste of such drugs to facilitate their formulation for oral administration.

Taste masking granules, however, is difficult. The use of granules contained in a sachet, in particular, taken by mouth and which releases drugs rapidly presents a taste-masking problem. The easiest way to mask a bitter taste is to mix a sweetener and flavor with the active ingredient, but many active ingredients are too bitter for masking this way. In general, the most effective way to taste mask a bitter active ingredient is to coat the granules. However, coated, large size granules have an uncomfortable mouth-feel. Some published papers have suggested that if pharmaceutical scientists desire to prepare granules with good mouth-feel, which are insoluble in the mouth, the particle size should be less than 100 μm. Wurster coating is the most suitable method for coating small particles, however it is very difficult to coat very small particles without agglomeration.

To solve the problem of taste masking for some bitter drugs, insoluble complexes of the drugs with ion exchange resins have been formed.

U.S. Pat. No. 5,032,393 teaches that the bitter taste of ranitidine may be masked by forming an adsorbate with a synthetic cation exchange resin. The adsorbate may then be incorporated into a composition for oral administration. Specifically, the synthetic cation exchange resin is selected from copolymers of styrene and divinylbenzene which are sulphonated, and copolymers of methacrylic acid and divinylbenzene.

U.S. Pat. No. 5,188,825 discloses a freeze-dried dosage form which is bonded to an ion-exchange resin to form a substantially water insoluble complex. Chlorpheniramine maleate and phenylephrine hydrochloride compositions are given as examples. Rhee et al., Yakhak Hoeji, 38(3):250-264 (1994) disclose an omeprazole-ion exchange resin complex wherein the particles of the complex are granulated into larger sized granules prior to coating with an enteric coating.

U.S. Pat. No. 3,951,953 discloses a semi-synthetic penicillin and anion exchange resin complex formulated as pharmaceutical compositions which can be in tablet or syrup form. The penicillin is said to be released rapidly and is said to be free of unpleasant taste.

U.S. Pat. No. 4,359,537 discloses fine particle size ion exchange resins, methods for their preparation, and the use of the particles to prepare emulsions.

WO 00/40224 discloses an oral dosage form containing a drug that is loaded onto an ion-exchange resin and subsequently coated.

However, in the case of particularly bitter unpleasant tasting drugs the use of such drug/resin complexes or adsorbates has not been sufficient to eliminate the undesirable taste. Additionally some ion-exchange resin materials themselves have unpleasant taste characteristics which add to, rather than reduce, the problems of formulating organoleptically acceptable oral dosage forms. Further, the ion exchange resins are not metabolized or absorbed by the body. The maximum amount of drug bound to ion-exchange resin is also limited. In the case of a high dose active ingredient, the final dosage form should be much bigger; therefore, it is not so easy for a patient to take it.

Therefore, a need exists for a taste-masked composition which masks the taste of particularly bitter or unpleasant tasting drugs. Such particularly bitter or unpleasant tasting drugs include nonsteroidal anti-inflammatory drugs including ibuprofen. The drugs are all weakly acidic materials.

Ibuprofen, a propionic acid derivative, is a nonsteroidal anti-inflammatory agent which possesses analgesic and antipyretic activity. Ibuprofen has been used in the treatment of rheumatoid arthritis, osteoarthritis, and mild to moderate pain. At present, many ibuprofen products are sold as over-the-counter medicines.

Ibuprofen has some undesirable properties, which are its bitter taste, low melting point and a tendency to sublime. These properties can cause development problems for some products. Attempts have been made to manufacture granules filled in a sachet by mixing several kinds of sweeteners and flavors with ibuprofen; however ibuprofen proved to be too bitter to prepare good tasting granules.

Another attempt was made to prepare coated granules employing several kinds of polymers. The bioavailability of these trial products was evaluated in humans or beagle dogs. The results showed that the products have equivalent bioavailability compared with commercial granules, coated with a porous membrane of ethylcellulose.

All of these products, including the commercial product, have disadvantages such as an uncomfortable mouth-feel and a change in taste that occurs over time due to sublimation of the drug. Therefore it has been shown that coated granules do not completely prevent sublimation.

It has been suggested that in order to prepare granules with good mouth-feel which are insoluble in the mouth, the particle size should be less than 100 μm. Wurster coating is the most suitable method for coating small particles, but agglomeration still occurs with very small particles. Prior to the present invention, only sugar-coated tablets or capsules had been shown to prevent sublimation. Other attempts have been made to prepare taste-masked granules of ibuprofen using other methods, such as spray drying, coacervation, phase separation, etc. Unfortunately, none of these preparations has a good taste, and they do not prevent sublimation.

Ibuprofen, as a carboxylic acid, can form salts with bases, such as basic amino acids, examples of which are lysine and arginine. The low solubility of the acid form of ibuprofen is overcome by the use of a lysine or arginine salt of ibuprofen. U.S. Pat. No. 4,279,926 describes the use of a lysine salt of ibuprofen in relieving pain and inflammatory conditions. However, the salt has a strong bitter taste and dissolves in the mouth, leaving a strong bitter taste. U.S. Pat. No. 5,200,558 describes L-amino acid and D-amino acid salts of S(+)-ibuprofen, a pharmaceutical composition, and a method for treating pain.

### SUMMARY OF THE INVENTION

One object of the present invention is to provide a complex of a drug having an unpleasant taste with polylysine or polyarginine, which has the ability to mask the taste of the drug and prevent sublimation.

Another object of the present invention is to provide a complex of a drug having an unpleasant taste with polylysine or polyarginine, which causes ions in saliva and gastric fluids to dissociate from the complex and provide good bioavailability.

Another object of the present invention is to provide a method for making a complex of a drug having an unpleasant taste with polylysine or polyarginine to prepare organoleptically acceptable granules, mask the taste of the drug, prevent sublimation and provide good bioavailability.

Another object of the present invention is to provide a complex of a drug having an unpleasant taste with polylysine or polyarginine as a way to prepare organoleptically acceptable granules, mask the taste of the drug, prevent sublimation and provide good bioavailability.

Another object of the present invention is to provide a complex of ibuprofen with polylysine or polyarginine as a way to prepare organoleptically acceptable granules, mask the taste of the drug, prevent sublimation and provide good bioavailability.

Another object of the present invention is to provide a taste-masked complex of ibuprofen with polylysine or polyarginine in tablet form.

### BRIEF DESCRIPTION OF THE DRAWINGS

FIG. 1 shows the dissolution profile of a polylysine-ibuprofen complex according to the present invention in distilled water, at pH 1.2 (HCl 0.07 mol, NaCl 0.03 mol) and at pH 6.8 (KH₂PO₄ 0.025 mol, Na₂HPO₄ 0.025 mol).

FIG. 2 shows the dissolution profile of an ibuprofen-lysine salt complex in distilled H₂O, at pH 1.2 (HCl 0.07 mol, NaCl 0.03 mol) and at pH 6.8 (KH₂PO₄ 0.025 mol, Na₂HPO₄ 0.025 mol).

FIG. 3 shows the dissolution profile of a polylysine-ibuprofen complex according to the present invention in NaCl solution.

FIG. 4 shows the dissolution profile of a polylysine-ibuprofen complex according to the present invention in KCl solution.

FIG. 5 shows the dissolution profile of a polylysine-ibuprofen complex according to the present invention in CaCl₂ solution.

FIG. 6 shows the dissolution profile of a polylysine-ibuprofen complex according to the present invention in phosphate buffer.

FIG. 7 shows a DSC thermogram of a polylysine-ibuprofen complex according to the present invention.

FIG. 8 shows a TGA thermogram of ibuprofen at 100° C. and 150° C.

FIG. 9 shows a TGA thermogram of polylysine-ibuprofen at 100° C. and 150° C. according to the present invention.

FIGS. 10 and 11a) and b) show physical properties of tablets of the invention.

FIGS. 12 and 13 show the dissolution profiles of tablets of formulations 1-5 of the invention.

FIGS. 14 and 15 show bioavailability data of ibuprofen in beagle dogs after oral administration.

FIG. 16 shows the dissolution profile of ibuprofen powder.

### DETAILED DESCRIPTION OF THE INVENTION

The present invention is related to pharmaceutical preparations for masking the unpleasant or bitter taste of drugs. The terms “masking the taste,” “tastemasking” or “taste masked” herein refer to reducing or eliminating the unpleasant or bitter taste of a drug and/or to improving of the taste of a drug.

The term “bitter taste” refers to a strong taste like black coffee without sugar, or very dark unsweetened chocolate.

The oral dosage form of the present composition comprises an insoluble complex of a drug with polylysine or polyarginine. The term “drug” used herein refers to a drug or an active ingredient, which is characterized by an unpleasant or bitter taste and which can bind to polylysine or polyarginine. Such drugs are usually strong or weak acids. For drugs having an unpleasant or bitter taste, the combination of complexing the drug with polylysine or polyarginine in accordance with the present invention provides good taste masking and facilitates the incorporation of the drug into the dosage forms for oral administration.

Drugs suitable for the claimed invention can have a bitter or unpleasant taste and are generally acidic and have a carboxyl group or are weak carbon acids such as piroxicam or phenylbutazone. Preferably the drug is a non-steroidal antiinflammatory drug (NSAID). More preferably the drug is selected from the group consisting of ibuprofen, ketoprofen, suprofen, ketoralac, carprofen, flurbiprofen, naproxen, fenoprofen, oxaprozin, etodolac, nabumetone, sulindac, tolmetin, piroxicam and phenylbutazone. Most preferably the drug is ibuprofen. Other representative drugs of unpleasant or bitter taste include, but are not limited to H₂ receptor antagonists, antibiotics, analgesics, cardiovascular agents, peptides or proteins, hormones, anti-migraine agents, anti-coagulant agents, anti-emetic agents, antihypertensive agents, narcotic antagonists, chelating agents, anti-anginal agents, chemotherapy agents, sedatives, anti-neoplastics, prostaglandins, antidiuretics and the like.

The oral dosage form of the present invention can be prepared by contacting the polylysine or polyarginine with the drug to form a drug/polylysine complex or a drug/polyarginine complex. Typically, polylysine or polyarginine suitable for use in the present invention is in the form of particles. Stirring the polylysine or polyarginine particles in a solution of the selected drug is usually sufficient to achieve binding of the drug to the polylysine or polyarginine particles. The drug/polylysine complex or drug/polyarginine complex may be further coated with a suitable coating material.

The nature of the bond between the drug and polylysine or polyarginine can be of any kind, such as ionic or chemical, as long as a salt is formed.

Ibuprofen forms a complex with the amino group of polylysine or polyarginine in a binding ratio of 1:1 based on the number of free amino groups on the polylysine or polyarginine. The maximum binding ratio is 1:1 based on the number of amino groups. Even if the ratio of ibuprofen is less than 1, the complexes have been sufficient to eliminate the unpleasant bitter taste.

The particle size of the drug/polylysine complex or the drug/polyarginine complex is less than 500 μm, and more preferably less than 100 μm. One advantage of the present invention is that particles less than 100 μm can be obtained having a good mouth feel. Some published results suggest that particle sizes larger than 100 μm give an unpleasant mouth-feel.

The oral dosage forms of the present invention can be used as a final oral dosage form and can be adapted for a range of final oral dosage forms including controlled release dosage forms. Suitable final dosage forms including controlled release dosage forms include, for example, suspensions, syrups, capsules, tablets, sprinkles, sachets, fast melt tablets, fast dissolving tablets and disintegrating tablet forms. The complexes according to the claimed invention may also be formulated in solid form which is reconstituted as a suspension prior to administration without losing any taste masking or prevention of sublimation properties.

The oral dosage forms of the invention may contain pharmaceutical additives, generally recognized as safe, in such amounts that they do not spoil the effects of the invention. Examples of such pharmaceutical additives include excipients, disintegrators, binders, lubricants, coloring agents, flavors, sweeteners, correctives, and the like.

When forming tablets of the inventive complex, the hardness of the tablets is greater than 15 kp regardless of the formulation, which means that the inventive complex has very good compressibility and the tablets will not chip or crack during distribution and transportation.

Preferably tablets of the inventive complex have a low ratio of the complex in the tablet to achieve good disintegration. If the complex ratio is too high, ibuprofen crystals may develop on the surface of the tablet and form a shell, thereby preventing disintegration. When the complex ratio is low, there is not enough ibuprofen to form a shell on the surface of the tablet and the presence of a large amount of excipients serves to prevent the build-up of ibuprofen crystals, therefore leading to faster disintegration times.

Dissolution profiles reflect disintegration time. Thus the tablets of the inventive complex also have good dissolution properties as indicated by the disintegration time.

The polylysine used in the present invention should preferably have a molecular weight greater than 2000. The inventive complex with low molecular polylysine has good solubility in distilled water and shows bitter taste. Therefore, the molecular weight of the polylysine should be greater than 2000 in order to precipitate and form a complex.

Tablets of the inventive complex have good bioavailability in animals and humans. Ions in the saliva of the mouth and gastric juices help to disassociate the complex, leading to good bioavailability as shown in the experiments conducted with beagle dogs.

Tablets of the inventive complex provide a good taste-masked oral dosage formulation because they do not dissolve in the mouth but show good bioavailability which is different from other taste-masking methods and products.

The following describes the invention further in detail with reference to inventive and test examples, but the invention is not limited to the following examples.

### EXPERIMENT

Materials

Ibuprofen

Ibuprofen (I) was obtained from BASF. Ibuprofen is a colorless, crystalline stable solid. Melting point is 75-77° C.

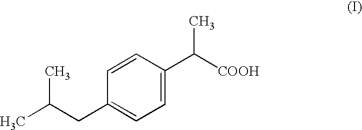

Polylysine

ε-Polylysine in its free base form (II) (MW:4700) was obtained from Chisso Corporation.

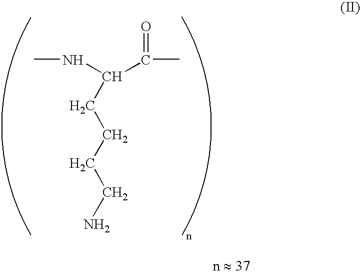

Poly-L-arginine Hydrochloride

Poly-L-arginine hydrochloride (MW:5000-15000) was obtained from Sigma.

Preparation of Complexes with Ibuprofen

Ibuprofen-Polylysine Complex

Ibuprofen (6.43 g) was dissolved in 20 mL-ethanol. Polylysine (4.0 g) was dissolved in 20 mL-distilled water. The ibuprofen solution was very clear. The polylysine solution was also clear, but it was slightly viscous and slightly yellow in color. Both solutions were mixed and shaken over night in a 50 mL-centrifugal tube. After mixing, a precipitate was formed. After shaking over night, the precipitate was separated by centrifugation. The precipitate was washed twice with 20 ml-ethanol, dried in a dessicator, ground and passed through a 60-mesh sieve.

Ibuprofen formed a complex with the amino group of polylysine in a binding ratio of 1:1 based on the number of free amino groups. The molecular weight of ibuprofen is 206. The molecular weight of one sub-unit of polylysine is 128. Based on these numbers, a mixing ratio of ibuprofen and polylysine was 1.61:1 by weight.

Sample

Ibuprofen-lysine Salt

Lysine free base (5.00 g) was dissolved in 20 mL-distilled water. This solution was very clear, and the pH was almost 10. Ibuprofen (7.05 g) was mixed and dissolved in the lysine solution. This solution was also very clear. Then the solution was dried in a vacuum dessicator to give a precipitate, and ground and passed through a 60-mesh sieve.

Ibuprofen-Polyarginine Complex

Polyarginine hydrochloride (100 mg) and sodium hydroxide (20.76 mg) were dissolved in 10 mL-distilled water, and then chloride and sodium ions were removed using a semi-permeable-membrane. Ibuprofen (106.7 mg) was dissolved in 25 ml-ethanol. Both solutions were mixed and shaken over night in a 50 mL-centrifugal tube. After mixing, a precipitate was formed. After shaking over night, the precipitate was separated by centrifugation. The precipitate was washed twice with 20 ml-distilled water, dried in a dessicator, ground and passed through a 60-mesh sieve.

Ibuprofen formed a complex with the amino group of polyarginine in a binding ratio of 1:1 based on the number of free amino groups. The molecular weight of ibuprofen is 206. The molecular weight of one sub-unit of polyarginine is 156. Based on these numbers, a mixing ratio of ibuprofen and polyarginine was 1.32:1 by weight.

In Vitro Dissolution Test

Complexes that corresponded to 150 mg of ibuprofen were subjected to dissolution in a USP (United States Pharmacopoeia) dissolution apparatus 2 (Paddle-type) with several kinds of solutions (900 ml). This dissolution test was conducted at 37° C., and a paddle rotation speed of 50 rpm was maintained. A UV detector was used to measure the absorbance of dissolution liquids at regular intervals of up to 120 minutes.

Thermal Analysis (DSC, TGA)

Differential Scanning Calorimetry Analysis (DSC)

Samples weighing approximately 2.5 mg were placed in aluminum pans and analyzed using Perkin-Elmer Thermal Analysis equipment DSC 7. The scanning speed was 10° C./min in range of 25-300° C.

Thermographic Analysis (TGA)

Samples weighing approximately 10 mg were placed in a platinum pan and the change in weight at a set temperature (100° C., 150° C.) was measured using Perkin-Elmer Thermal Analysis Equipment.

Sensory Taste Test

A taste test of bitterness was carried out using the inventive complexes. The taste test was carried out by a panel of four members, by keeping each complex in the mouth for about 20 seconds and then judging the degree of bitterness based on the following evaluation criteria. The results are shown in Table 1.

A: No bitter taste.

B: Almost no bitter taste.

C: Slightly bitter taste.

D: Bitterness taste.

E: Strong bitter taste.

TABLE 1

| Panelist | 1 | 2 | 3 | 4 |
|---|---|---|---|---|
| Ibuprofen-polylysine complex | A | A | B | A |
| Ibuprofen-polyarginine complex | A | A | A | B |
| Ibuprofen-Polylysine complex | A | A | B | B |
| after 2 months storage | | | | |
| Ibuprofen-Polyarginine complex | B | A | B | A |
| after 2 months storage | | | | |
| Ibuprofen-lysine salt complex | E | E | E | E |

Although the evaluation method for taste was subjective, as can be seen from the results above, the inventive complex had good taste masking compared to ibuprofen powder or ibuprofen-lysine salt. In general, some ibuprofen products, including the commercial product, change taste after storing for 1-2 months under room temperature. This change in taste may be because of sublimation of ibuprofen. However the inventive polylysine-ibuprofen and polylysine-arginine complexes did not exhibit any change in taste after storage for the same amount of time under the same conditions.

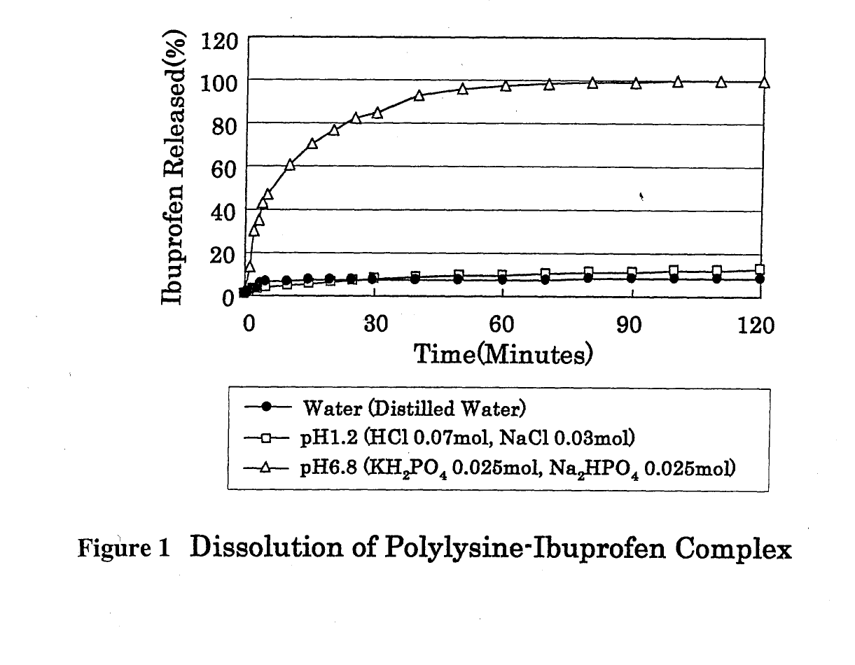

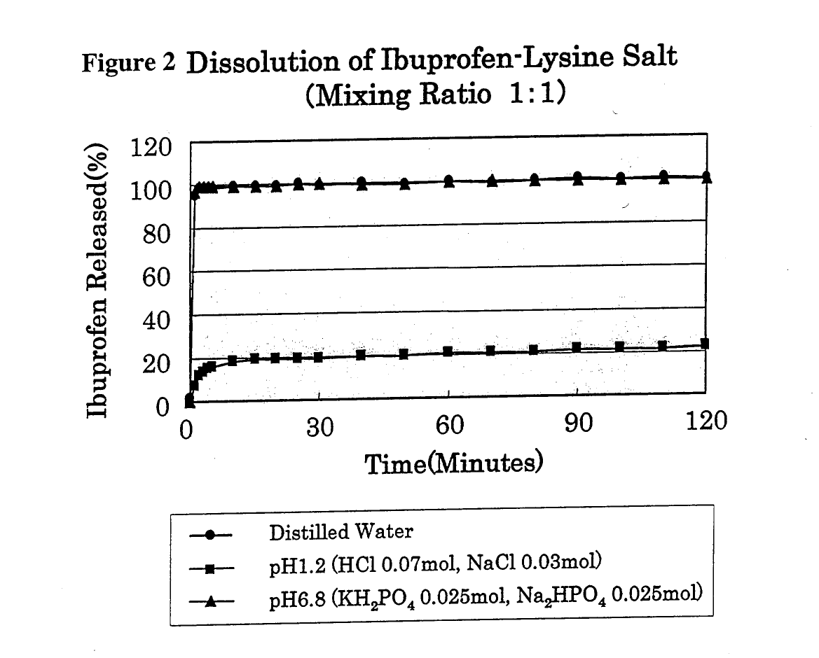

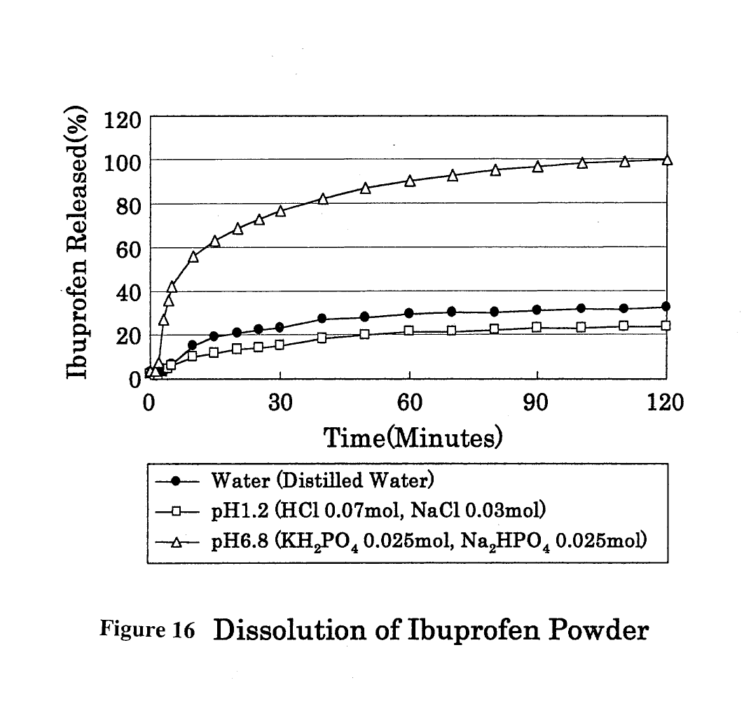

FIGS. 1 and 2 show dissolution profiles in water (distilled water), at pH 1.2 (HCl 0.07 mol, NaCl 0.03 mol), and at pH 6.8 (KH₂PO₄ 0.025 mol, Na₂HPO₄ 0.025 mol). The polylysine and ibuprofen complex has very poor solubility in distilled water, whereas the solubility of the ibuprofen-lysine salt complex is high in distilled water. Even though the solubility of ibuprofen in water at low pH is not good, the dissolution profile of ibuprofen in distilled water would saturate at 40% or so. (FIG. 16). In short, the solubility of the complex in distilled water is much poorer than the solubility of ibuprofen in water. The dissolution profile of the polylysine-ibuprofen complex in pH 1.2 media was much lower. It was observed, however, that a white substance formed and adhered to the paddle as soon as the complex was put in the dissolution test liquid. The white substance was confirmed to be ibuprofen crystals. Therefore, dissociation of the complex and crystallization of ibuprofen were very fast. If a large ibuprofen crystal formed in the human intestine, the bioavailability would be expected to be very poor.

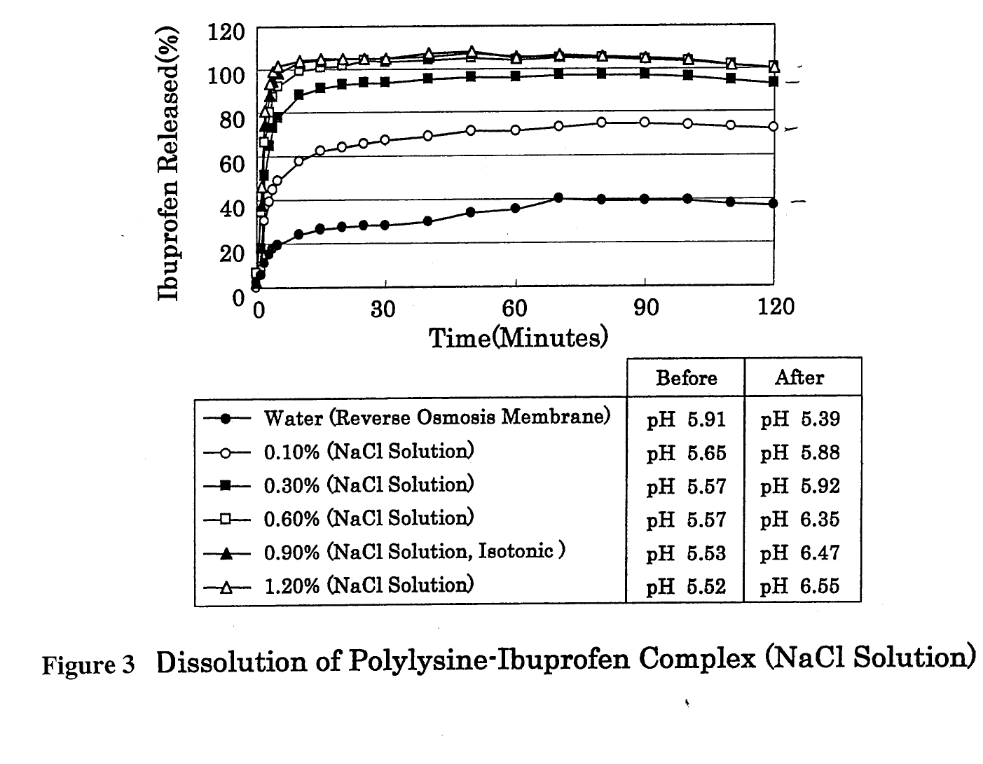

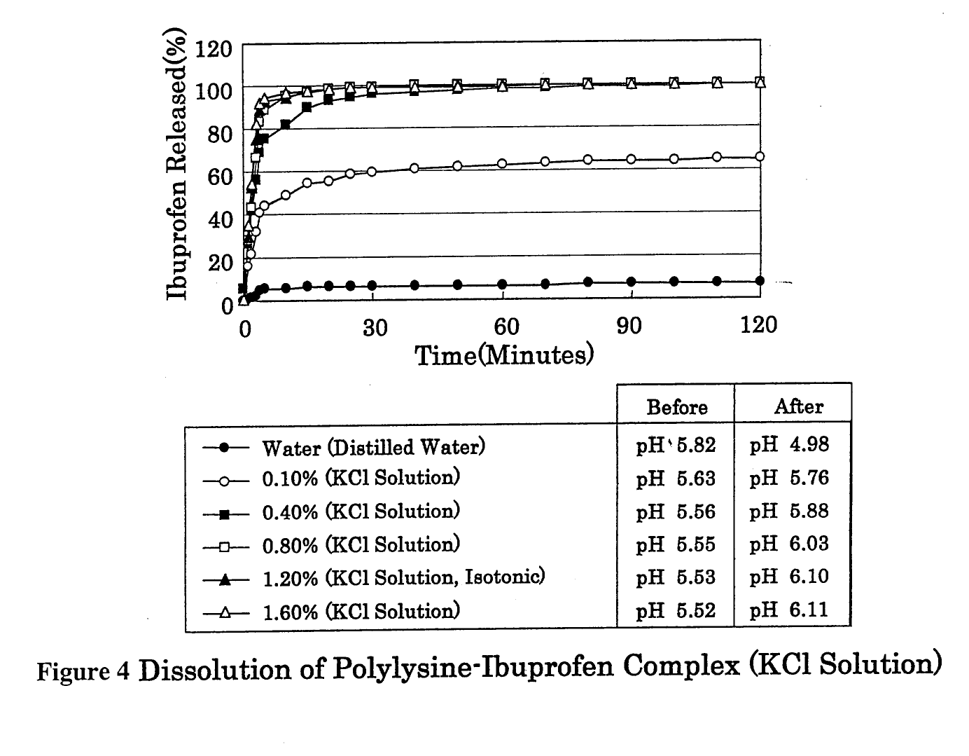

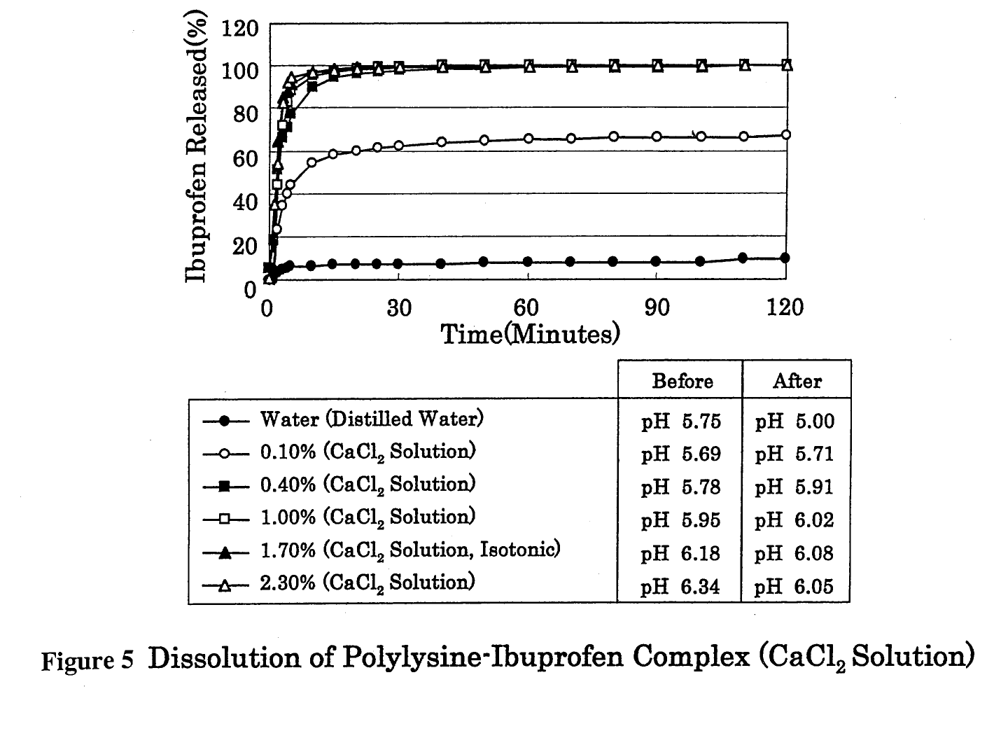

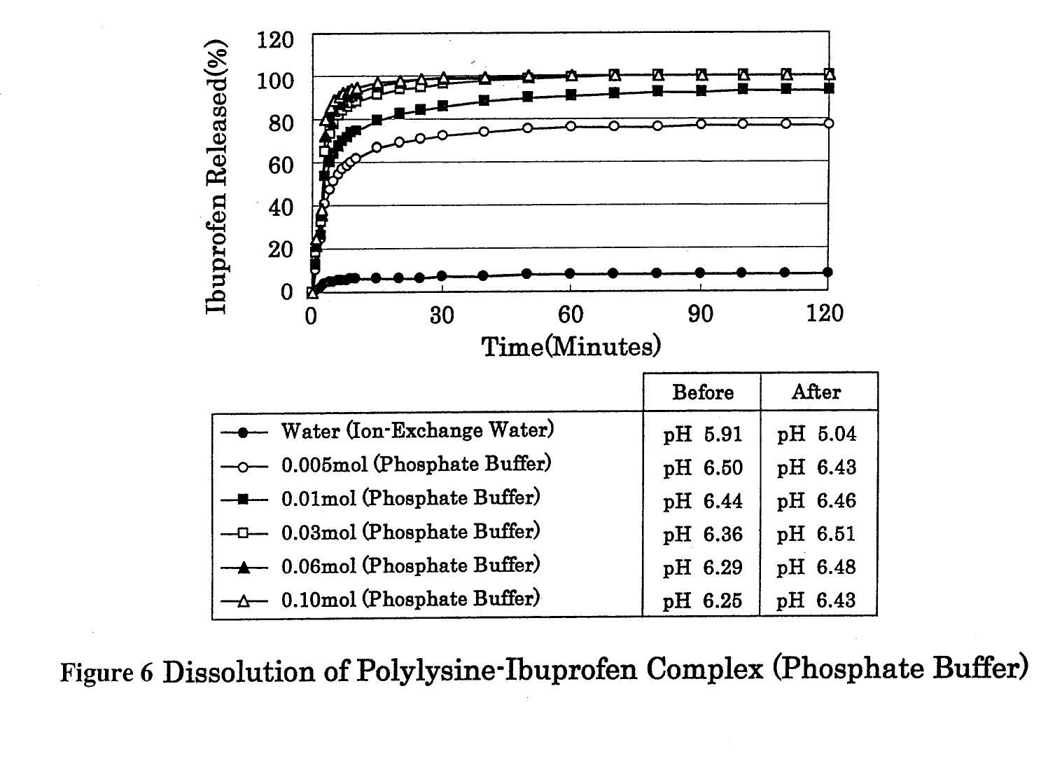

FIGS. 3-6 show other dissolution profiles for the polylysine-ibuprofen complex. Each figure shows almost the same profile. The complex dissolves at the same rate regardless of the kind and the concentration of ions. These experiments used three kinds of water, distilled water (FIGS. 4 and 5), ion-exchange water (FIG. 6) and reverse osmosis membrane water (FIG. 3). In the case of using reverse osmosis membrane water, the dissolution profile was different compared to other data. This is most likely because the reverse osmosis membrane water included some ions.

From these data, it can be seen that complete taste masking may be difficult, because there will be some ions in tap water and saliva which will dissociate the complex and lead to a bitter taste. But, saliva may not cause a strong bitter taste because the amount of saliva in the mouth is very small and the osmotic pressure should be isotonic, which means the concentration of ions in saliva is not high. Taking a medicine with tap water should not lead to a strong bitter taste because the medicine is only in the mouth for a very short time.

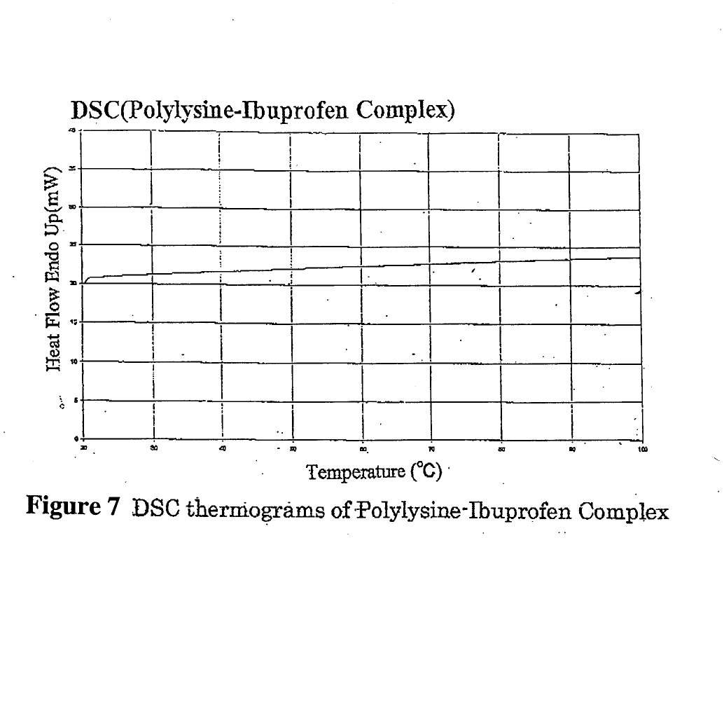

FIG. 7 shows a DSC thermogram of a polylysine-ibuprofen complex. Although ibuprofen has a melting point between 75-77° C., the complex does not show a melting point. This means that the complex has an advantage in manufacturing processes by preventing ibuprofen powder built up on the inside wall of the granulation chamber and on the punch apparatus of the tabletting machine. This physical property of these complexes can prevent such troubles in the manufacturing process.

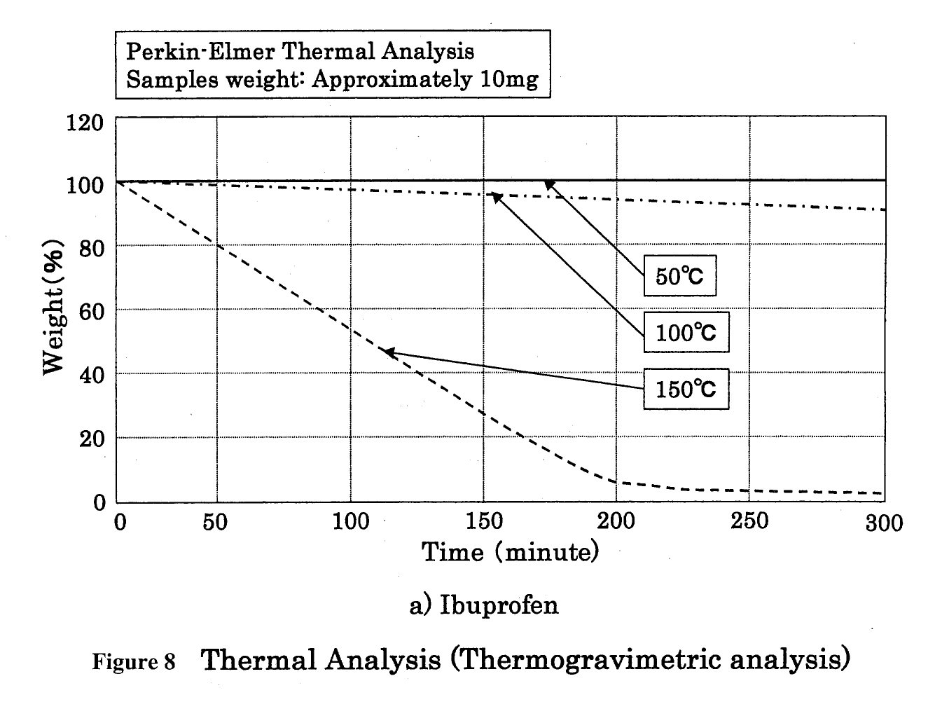

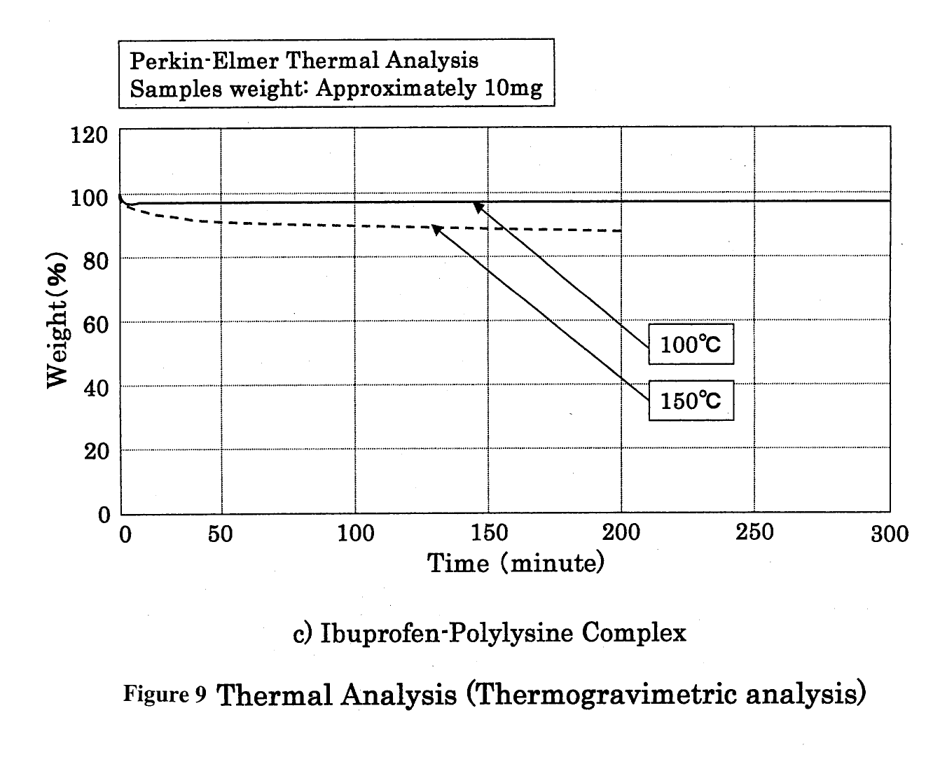

FIG. 8 is a TGA thermogram of ibuprofen. FIG. 9 is a TGA thermogram of the polylysine-ibuprofen complex. It is well known that ibuprofen can sublime at room temperature. The TGA data for ibuprofen showed sublimation at both 100° C. and 150° C., although there is a much greater change over time at 150° C. It was observed that some ibuprofen products, including the commercial product, change taste after storing for 1-2 months under room temperature. These changes in taste may be due to sublimation of ibuprofen. TGA data of ibuprofen also supported this conclusion. On the other hand, the polylysine-ibuprofen complex was very stable at 100° C. and the weight decrease at 150° C. was much smaller than it was for Ibuprofen. Thus the polylysine-ibuprofen complex should prevent sublimation and prevent a changing of taste overtime.

Preparation of Tablets

The ibuprofen-polylysine complex was mixed with microcrystalline cellulose and a disintegrator (Ac-Di-Sol or Explotab) for 10 minutes in a bottle. Then, magnesium stearate was added and mixed for 3 minutes. All formulations are described in Table 2. In the formulations, 121 mg of complex is equivalent to 75 mg of ibuprofen, i.e., 1 tablet includes 75 mg ibuprofen.

TABLE 2
Formulations

| | | | | | Total | Ratio of |
|---|---|---|---|---|---|---|
| | Complex | MC | Ac-Di-Sol | Mg-St | weight | complex in |
| No. | (mg) | (mg) | (mg) | (mg) | (mg) | a tablet (%) |
| 1 | 121 | 120 | 12.1 | 0.63 | 254 | 47.7 |
| 2 | 121 | 90 | 10.6 | 0.55 | 222 | 54.5 |
| 3 | 121 | 60 | 9.1 | 0.48 | 191 | 63.5 |
| 4 | 121 | 30 | 7.6 | 0.40 | 159 | 76.1 |
| 5 | 121 | 15 | 6.8 | 0.36 | 143 | 84.5 |
| 6 | 121 | 85 | 15.5 | 0.55 | 222 | 54.5 |
| 7 | 121 | 80 | 20.1 | 0.55 | 222 | 54.6 |
| 8 | 121 | 72 | 29.0 | 0.55 | 223 | 54.4 |
| | — | — | Explotab | — | — | — |
| 9 | 121 | 90 | 10.6 | 0.55 | 222 | 54.5 |
| 10 | 121 | 85 | 15.5 | 0.55 | 222 | 54.5 |

The mix was then compressed with a 5/16 inch standard concave punch using a single tabletting machine. Compression pressure was 0.5 or 1.0 ton.

Quantitative Analysis

Quantitative analysis of the complexes was carried out using an ultraviolet detector (UV, wave-length was 263 nm). The complexes were dissolved or dispersed in distilled water (100 ml volumetric flask) with a 5 mL-sodium hydroxide solution (1N). Then the UV absorbance was measured.

Disintegration Time Test

Disintegration times were determined using a USP method, as described on the USP monograph.

Measuring Hardness of Tablets

The hardness of the tablets was measured using a Tablet Hardness Tester (Delamar, Inc. model No. PT1000).

In Vitro Dissolution Test

The complexes containing 150 mg of ibuprofen were subjected to dissolution testing using a USP dissolution apparatus 2 (Paddle-type) with several kinds of solutions (900 ml). This dissolution test was conducted at 37° C., at a paddle rotation speed of 50 rpm. A UV detector was used to measure the absorbance of dissolution liquids at regular intervals up to 120 minutes.

Bioavailability Test with Beagle Dogs

Products

The trial sample is formulation No. 6 in Table 2. This tablet includes 121 mg ibuprofen-polylysine complex, which means that the tablet has 75 mg of ibuprofen. A commercial product (NUROFEN) produced by Taisho Pharmaceutical Co., Ltd. was used in this experiment as a reference. This commercial product also includes 75 mg of ibuprofen. Although the commercial product is a film-coated tablet, it has shown good results in human bioavailability tests.

Subjects

Eight healthy male beagle dogs were used in this experiment after an overnight fast. This experimental design was a crossover fashion.

Dosing and Blood Sampling

Following an overnight fast, beagle dogs took one tablet (trial sample or commercial product). A blood sample (4 ml) was taken intravenously according to the following time schedule.

Before dosing

After dosing: 20 minutes, 40 minutes, 1 hour, 1.5 hours, 2 hours, 3 hours, 4 hours, 6 hours and 12 hours.

Assay of Ibuprofen in Plasma

Ibuprofen was assayed in plasma using high performance liquid chromatography (HPLC).

Treatment of Data

Pharmacokinetic parameters were calculated using standard equations for ibuprofen. Typically, after calculating the elimination rate on the plasma concentration-time curve, the AUC (area under the curve) would be calculated under the time range from 0 to infinity. But, in this calculation of the AUC of ibuprofen, the time range was from 0 to 12 hours. Also, statistical analysis was carried out using ANOVA (Analysis of Variance).

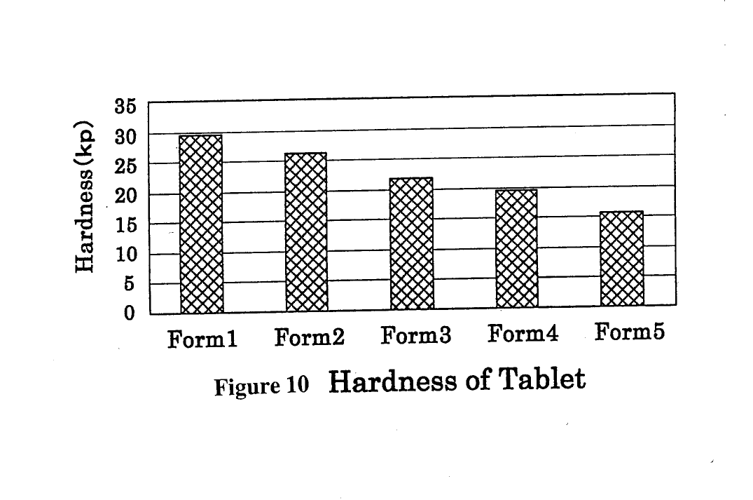

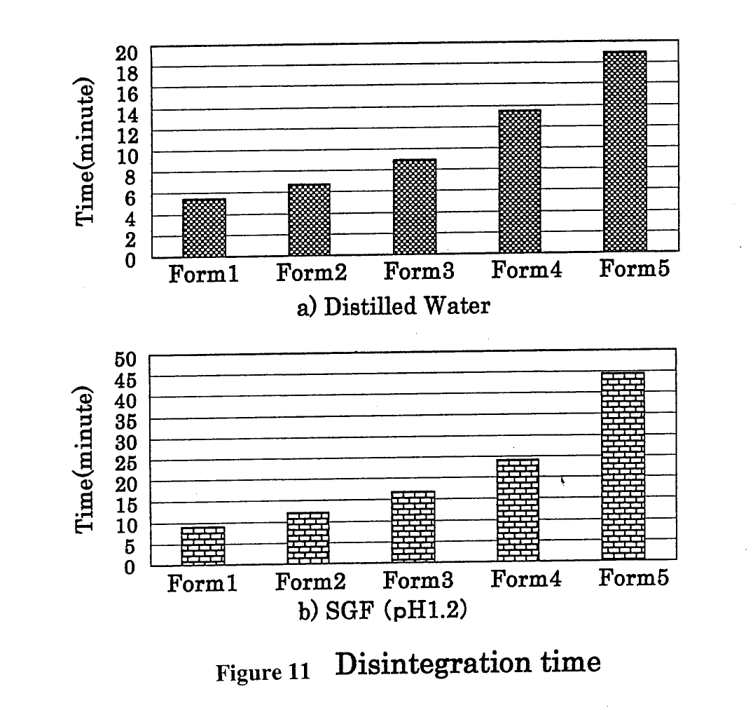

FIGS. 10 and 11 show the physical properties of tablets of Formulations 1-5. In the case of making tablets using a single punch machine, the compression time was long compared to that for an ordinary tabletting machine. Actually, the hardness of tablets of Formulations 1-5 compressed using a single tabletting machine was very high. Although long-term compression helps to make harder tablets, the compressibility of the complex is very good.

The hardness of the tablets also depends on the formulation. Formulation 1 was harder than the other tablets. Hardness also depends on the thickness of the tablets. In these experiments all of the tablets were produced using the same punch, which means that the thickness was due to the formulation. In fact, Formulation 1 was thicker than the other tablets.

FIGS. 11a) and b) show the disintegration time of tablets of Formulations 1-5. Disintegration time tests were carried out using two kinds of liquid, distilled water and simulated gastric fluid (SGF). In the case of distilled water, microcrystalline cellulose and disintegrator (Ac-Di-Sol) have a big influence on the disintegration time because the complex cannot dissolve in distilled water. Also a lower concentration of the complex in the tablet leads to a shorter disintegration time. Tablet disintegration in SGF liquid was longer independent of the formulation. In fact, the ibuprofen-polylysine complex can dissolve in SGF and the dissociation rate is very fast as indicated by the results of the dissolution test. In the dissolution test using SGF (FIG. 1), a substance with large white crystals appeared after rapid dissociation. The crystals were ibuprofen crystals. Therefore, it is expected that the same crystallization behavior also occurs in the disintegration time test. In the case where the concentration of the complex in the tablet is high, the surface of the tablet changes to ibuprofen crystals and the crystals form a shell on the surface of the tablet as soon as the disintegration time test is started. This shell prevents disintegration of the tablet. When the concentration of the complex in the tablet is low, there are not enough ibuprofen crystals to form a shell on the surface of the tablet. Also, a large amount of excipients present prevents the build-up of ibuprofen crystals. As a result, disintegration time is shorter.

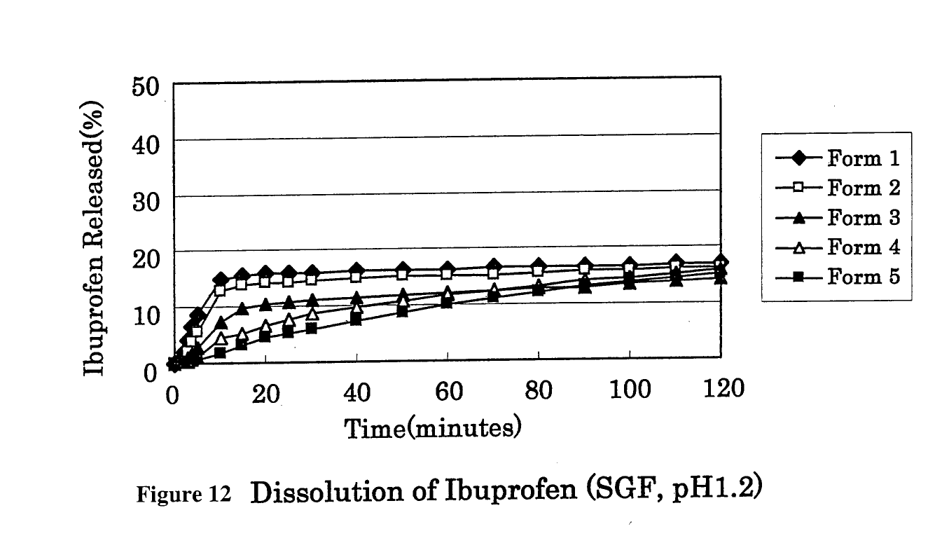

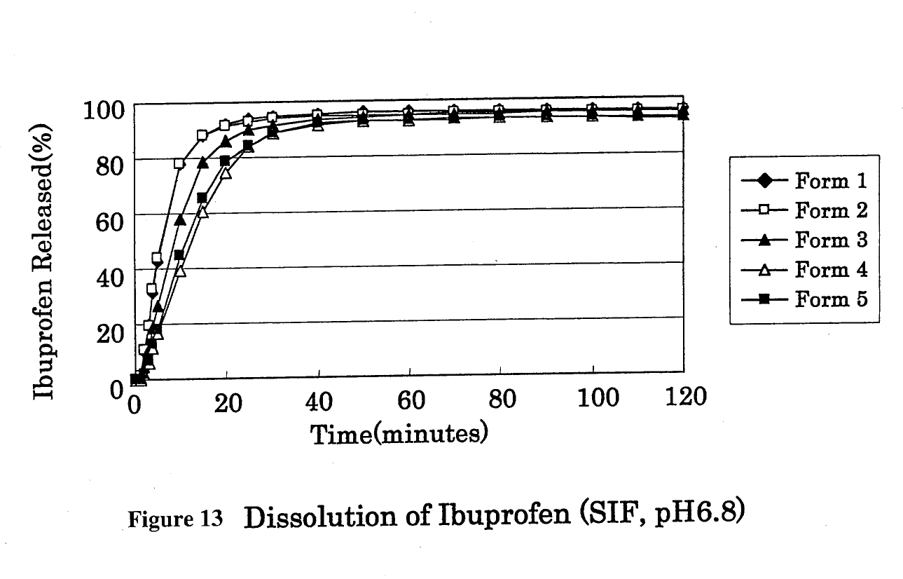

FIGS. 12 and 13 show dissolution profiles of tablets of Formulations 1-5. Dissolution profiles reflect disintegration time. Formulations 1 and 2 have very good dissolution profiles in both liquids. As mentioned above, the complexes produced large ibuprofen crystals after putting them into SGF to start the disintegration time tests and the dissolution profiles were not especially good. However, in the case of Formulations 1 and 2, the dissolution profiles were better because some of the excipient prevented the growth of large ibuprofen crystals. In other formulations, big ibuprofen crystals were formed in SGF during the dissolution test. These tablets did not have enough excipient to prevent the growth of ibuprofen crystals.

Tablets of formulations 6-10 were prepared under the same conditions. Formulations 6-8 had a little bit faster disintegration time than Formulation 1, wherein the compression force was changed from 1.0 ton to 0.5 ton, because previous tablets were too hard. As a result, the disintegration times of Formulations 6-8 were less than 75 seconds in distilled water and their hardness was approximately 10 kp. The disintegrator was changed from Ac-Di-Sol to Explotab in Formulations 9 and 10. Explotab did not work well; and the disintegration times of Formulations 9 and 10 were more than 10 minutes in distilled water. Finally, Formulation 6 was selected for the bioavailability tests since the results of the dissolution test with Formulation 6 were almost the same as with Formulations 1 and 2.

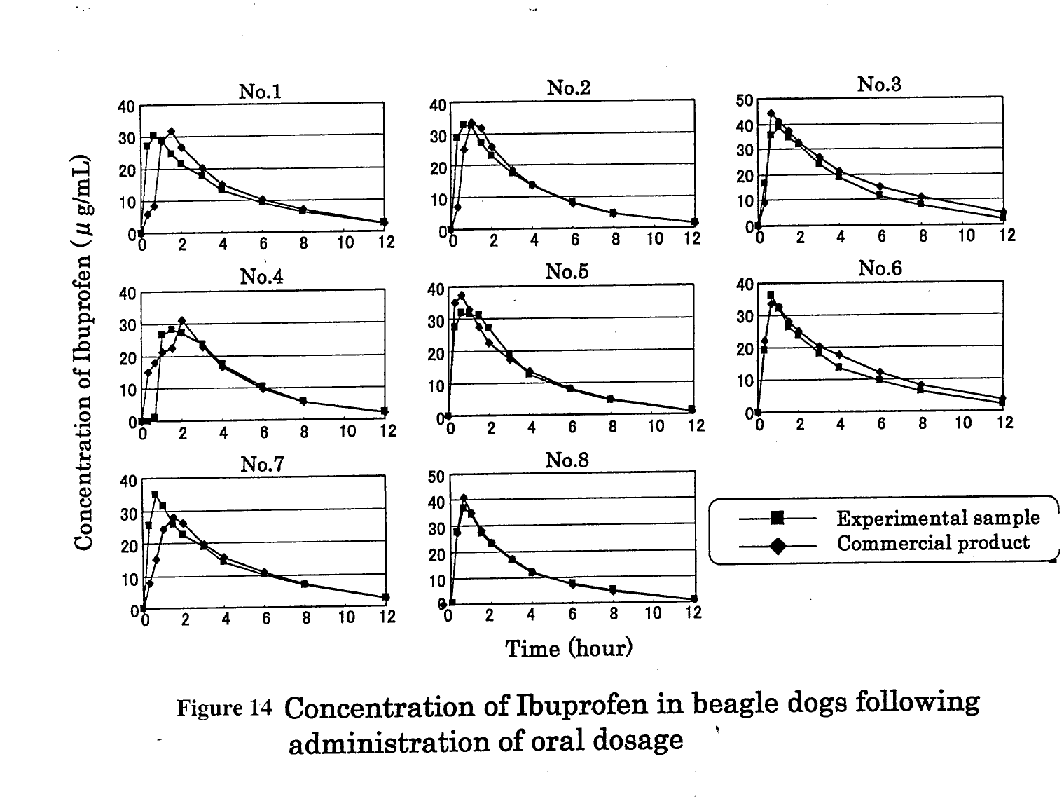

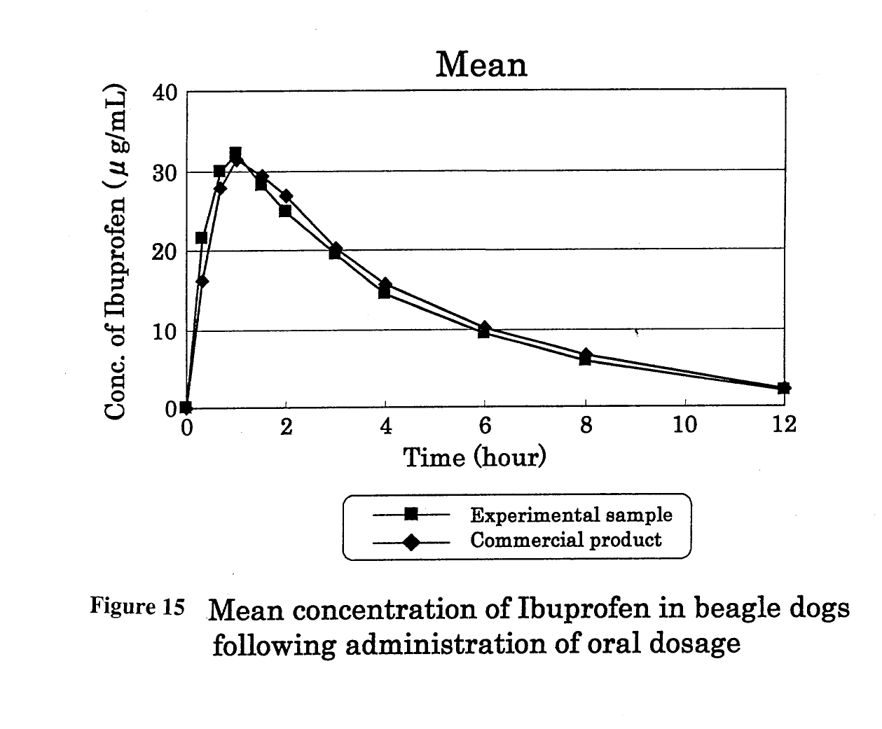

FIGS. 14 and 15 show bioavailability data in beagle dogs following administration of the oral dosage forms. Table 3 shows the bioavailability indices. As mentioned before, based on the results of the various dissolution tests, it was expected that the dissociation rate would be very fast in saliva and gastric juice, and bioavailability would be good. Actually bioavailability data of the trial sample was good, because the trial sample and commercial product showed equivalent bioavailability. The commercial sample used in this study showed good bioavailability in human absorption tests in a previous study and is currently available on the market. According to bioavailability indices, Tₘₐₓ of Formulation 6 was a little bit faster (not a significant difference) than the commercial product. Although this difference is not significant, it indicates that Formulation 6 of the present invention will show a faster Tₘₐₓ in human bioavailability tests, because all of the complex did not dissociate in the stomach of the beagle dog, which is smaller than a human stomach.

The present work studied the complex of ibuprofen with polylysine and a complex with ibuprofen and polyarginine. Complexes with polylysine and with polyarginine were prepared and the complexes showed good physical properties, good taste, and good resistance to sublimation at a high temperature. Regarding the dissolution profiles, the presence of ions, regardless of the kind of ion, stimulated dissociation of the complex, which is quiet fast. Therefore, these complexes show good bioavailability and appear to have the best properties for an ibuprofen complex product.

TABLE 3
Bioavailability Indices

| | | | | |
|---|---|---|---|---|
| | Cmax | AUC_{0-12 h} | Tmax | MRT_{0-12 h} |
| Animal | (ng/mL) | (ng · hr/mL) | (hr) | (hr) |

Commercial Product (Tablet)

| | | | | |
|---|---|---|---|---|
| No.1 | 31.85 | 142.36 | 1.50 | 4.10 |
| No.2 | 33.84 | 130.46 | 1.00 | 3.46 |
| No.3 | 44.67 | 211.12 | 0.67 | 4.16 |
| No.4 | 31.12 | 142.75 | 2.00 | 3.83 |
| No.5 | 37.32 | 139.21 | 0.67 | 3.27 |
| No.6 | 33.53 | 167.49 | 0.67 | 4.03 |
| No.7 | 27.86 | 143.70 | 1.50 | 4.14 |
| No.8 | 41.04 | 134.74 | 0.67 | 3.13 |
| Mean | 35.15 | 151.48 | 1.09 | 3.77 |
| S.D. | 5.54 | 26.47 | 0.52 | 0.42 |
| S.E. | 1.96 | 9.36 | 0.18 | 0.15 |

Experimental Sample Ibuprofen-Polylysine Complex

| | | | | |
|---|---|---|---|---|
| No.1 | 30.41 | 138.61 | 0.67 | 3.76 |
| No.2 | 33.02 | 134.78 | 0.67 | 3.32 |
| No.3 | 39.11 | 179.19 | 1.00 | 3.73 |
| No.4 | 28.11 | 137.98 | 1.50 | 4.08 |
| No.5 | 31.94 | 137.54 | 0.67 | 3.23 |
| No.6 | 36.35 | 144.56 | 0.67 | 3.73 |
| No.7 | 34.79 | 149.52 | 0.67 | 3.82 |
| No.8 | 36.66 | 133.68 | 0.67 | 3.28 |
| Mean | 33.80 | 144.48 | 0.82 | 3.62 |
| S.D. | 3.62 | 14.96 | 0.30 | 0.31 |
| S.E. | 1.28 | 5.29 | 0.11 | 0.11 |

What is claimed is:

1. A taste-masked oral dosage form comprising a complex of a drug with polylysine or polyarginine.

2. The taste-masked oral dosage form according to claim 1, wherein the drug has a bitter taste.

3. The taste-masked oral dosage form according to claim 1, wherein the drug is acidic and has a carboxyl group.

4. The taste-masked oral dosage form according to claim 1, wherein the drug is a propionic acid derivative compound.

5. The taste-masked oral dosage form according to claim 1, wherein the drug is a non-steroidal anti-inflammatory agent.

6. The taste-masked oral dosage form according to claim 1, wherein the drug is selected from the group consisting of ibuprofen, ketoprofen, suprofen, ketoralac, carprofen, flurbiprofen, naproxen, fenoprofen, oxaprozin, etodolac, nabumetone, sulindac, tolmetin, piroxicam and phenylbutazone.

7. The taste-masked oral dosage form according to claim 1, wherein the drug is ibuprofen.

8. The taste-masked oral dosage form according to claim 1, wherein the drug has a particle size less than about 100 μm.

9. The taste-masked oral dosage form according to claim 1, wherein the drug and said polylysine or polyarginine are bound in a 1:1 ratio based on the number of free amino groups of polylysine or polyarginine.

10. The taste-masked oral dosage form according to claim 1, wherein the complex prevents sublimation of the drug.

11. The taste-masked oral dosage form according to claim 1, wherein changing of the taste of the drug over time is prevented.

12. The taste-masked oral dosage form according to claim 1, which is a controlled release dosage form selected from the group consisting of suspensions, syrups, dry syrups, capsules, tablets, sprinkles, sachets, fast melt tablets, fast dissolving tablets and disintegrating tablet forms.
